(19) United States
(12) Patent Application Publication
(10) Pub. No.: US 2025/0018212 A1
(43) Pub. Date: Jan. 16, 2025

(54) APPARATUS AND METHOD FOR WEARABLE ELECTRON PARAMAGNETIC RESONANCE IMAGING AND MAGNETICALLY-ENABLED THERAPY

(71) Applicant: Weinberg Medical Physics, Inc., North Bethesda, MD (US)

(72) Inventors: Irving N. WEINBERG, North Bethesda, MD (US); Oleg UDALOV, North Potomac, MD (US)

(21) Appl. No.: 18/441,692

(22) Filed: Feb. 14, 2024

Related U.S. Application Data
(60) Provisional application No. 63/445,532, filed on Feb. 14, 2023.

(51) Int. Cl. A61N 2/02

(52) U.S. Cl. CPC A61N 2/02

(57) ABSTRACT

An apparatus and method are provided for collecting data about the free electron density of a sample using an array of coils. The apparatus and method provide for the array of coils to be lightweight and wearble.

### CROSS-REFERENCE AND PRIORITY CLAIM

This application claims priority to U.S. Provisional Patent Application Ser. No. 63/445,532, entitled “APPARATUS AND METHOD FOR WEARABLE ELECTRON PARAMAGNETIC RESONANCE IMAGING AND MAGNETICALLY-ENABLED THERAPY” filed Feb. 14, 2023, the entirety of which is incorporated by reference.

### FIELD

A method and apparatus are provided for diagnosing and treating diseases in animals, including humans. The method and apparatus may be used in medical and veterinary applications. The method and apparatus may also be used outside of medicine, for example in parts inspection.

### BACKGROUND

Magnetic resonance imaging (MRI) of protons in the body is useful for the depiction of soft-tissue injuries and structures. Effective MRI requires that the spins of a majority of the species to be imaged (e.g., protons, electrons) are initially polarized (i.e., oriented in a specific direction). The direction of initial polarization is generally parallel to the static magnetic field created by the MRI, in which the static field is often called “BO”. In the course of a typical MRI pulse sequence, the spins of the species to be imaged are tipped from this initial polarization direction through the judicious application of radiofrequency (RF) pulses and/or the application of transient magnetic fields in a direction perpendicular to BO. In general, the magnitudes of the transient magnetic fields vary in space, and such fields are generally called “magnetic gradients”. A classic MRI pulse sequence involving the use of repeated magnetic gradients is known as echo planar imaging. Other classic MRI pulse sequences combine both RF and gradients to form images. In some types of MRI systems (i.e., fast-cycling MRI), the magnetic field used to polarize the protons is different than the so-called evolution magnetic field which is used in the imaging part of the pulse sequence.

For protons in liquids, it takes about a second for the spins to orient along the BO magnetic field. The time required to align the spins in the BO direction is called T1. Most MRI studies take tens of minutes to complete. Creating a static magnetic field that lasts for such a long period, and which has sufficient strength to align large numbers of proton spins, requires coils through which a great deal of electrical current flows (often tens of thousands of amperes). Conventional electromagnet coils would melt if such high currents were imposed for periods longer than a few seconds. Most MRI systems use superconducting wires in their electromagnet coils to prevent the long-lasting high currents from melting the coil wires. These superconducting electromagnets require expensive pumping and cooling systems, and take up lots of space.

Beside protons, there are other spin carriers in living tissues, for example free electrons. Free electrons have T1 times that are about a million times shorter than for protons. MRI with free electrons is called electron paramagnetic resonance imaging (EPRI). Since the T1 for free electrons is so short, total MRI time could theoretically be much shorter than in proton MRI.

The decay of the RF signal from electrons (T2) is also much shorter than for protons. For free electrons in tissue, T1 and T2 are typically on the order of microseconds. Certain molecules (“spin traps”) may be used to effectively increase T1 and T2 of free electrons.

It is known that EPRI can be performed with the continuous application of RF to a sample, while the sample resides in a slowly-varying quasi-static magnetic field created by a heavy resistive coil that is water-cooled. An example of this technique is described in the publication by Hiroshi Hirata, Guanglong He, Yuanmu Deng, Ildar Salikhov, et al, entitled “A loop resonator for slice-selective in vivo EPR imaging in rats”, Journal of Magnetic Resonance 190 (2008) 124-134. A typical resistive coil apparatus for EPRI with a 23-mm bore weighs hundreds of kilograms (e.g., Bruker Elexys-II-E450).

It is known that EPRI can be performed with pulsed application of RF to a sample if the static field is supplied by a very stable magnet, for example the fringe field of a superconducting MRI, as taught by A. Feintuch, G. Alexandrowicz, T. Tashma, et al, in the publication entitled “Three-Dimensional Pulsed ESR Fourier Imaging”, Journal of Magnetic Resonance 142, 382-385 (2000)).

In both of the above examples, RF energy is deposited in the sample. Such deposition can heat and thereby damage tissues. An alternative approach to collecting data from a sample is to create spin echoes using alternating magnetic gradients, a technique known as gradient echo imaging. A popular pulse sequence that uses this principle is known as echo planar imaging.

### SUMMARY

Disclosed embodiments describes the design and use of a novel system and method for EPRI. The same system may also be used to deliver therapy.

Disclosed embodiments utilize non-superconducting electromagnets (i.e., resistive coils) to create the short-acting magnetic fields needed to collected images with free electrons.

A compact and lightweight apparatus and method are disclosed for implementing EPRI that involves the use of coils to generate a polarizing magnetic field and magnetic gradients. Said polarizing magnetic fields and gradients may be generated with lightweight coils in a wearable helmet or other compact configuration. The time-scales of the polarizing and gradient fields vary in time-scales comparable to the electron T1 time. Disclosed embodiments accomplish imaging, without the need for superconducting or permanent magnets and without depositing RF energy in a subject. The system coils used to deliver the magnetic fields for EPRI may be so lightweight as to be wearable. The lightweight system may also be used to deliver therapy, for example by opening a blood-brain barrier to drugs or by driving magnetic tools.

### BRIEF DESCRIPTION OF THE FIGURES

FIG. 1 shows an embodiment of an apparatus for image collection and intervention;

FIG. 2 illustrates a flowchart of a method according to the disclosed embodiments;

FIG. 3 illustrates a function of an apparatus according to the disclosed embodiments; and

FIG. 4 shows the results of a simulation of EPRI according to the disclosed embodiments.

### DETAILED SPECIFICATION

Disclosed embodiments describe an apparatus and method utilizing coils and power supplies to collect images of free electron density in a sample and potentially to intervene based on said images. The sample may be a living human or non-human animal's brain or heart or some other body part.

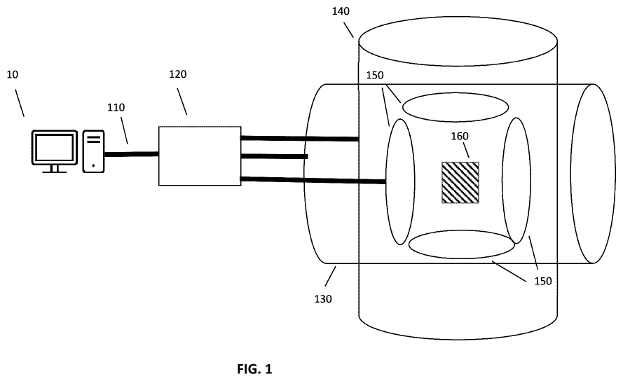

As shown in FIG. 1, the apparatus an apparatus 10 may be controlled by a computer or processor 100 which communicates via wires 110 or wirelessly (not shown) to control a source of electrical power source 120. It is understood that electrical power source 120 may contain or be connected to a battery or generator and may have internal boost capability to generate electrical pulses that may be as high as 1000 amperes for durations of 0.1-10 microseconds in a repeated fashion. One or more power sources 120 may be connected with a cable to a coil assembly 130 that generates a polarizing magnetic field. The one or more power sources 120 may also connected with a cable to a coil assembly 140 that generates evolution magnetic fields, generally in an orthogonal direction to the polarizing field. The one or more power sources 120 may also be connected with a cable to a coil assembly 150 that generates gradient magnetic fields. The power source 120 may also connected with a cable to a coil assembly (not shown) that generates and/or measures RF energy into and/or from a sample 160. The sample may be a human body part (for example, a brain or heart). The orientations, sizes, and configurations of the coil assemblies in FIG. 1 are illustrative and not to scale. Additionally, some functions of the coil assemblies may be performed or replaced by other coil assemblies. For example, one or more of the gradient coils 150 may carry polarizing current to replace the need for a polarizing coil 130.

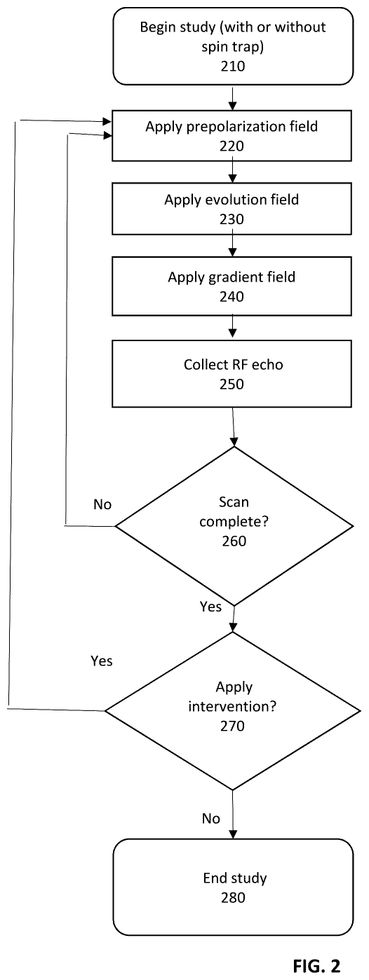

According to FIG. 2, a method similar to an echo planar imaging pulse sequence may be provided. A study (which may be purely diagnostic or may include an intervention) may be initiated 210. Depending on the scenario, a spin trap may be administered to the subject at this time or at other times in the sequence of events. In operation 220, a polarizing field may be applied by a polarizing coil, for example in the X direction. A smaller evolution field in an orthogonal (BO) direction may be applied while the polarizing coil is off 230, or may be applied throughout the polarizing field operation 220. Magnetic gradients may be applied 240. In operation 250 which may occur during part of operation 240, RF acquisition of echoes may proceed. Each echo acquisition provides data that may be used to reconstruct an image. In most cases, multiple iterations of operations 220 to 250 are needed to collect a high quality image. If the number and quality of steps is assessed inadequate 260, then operations 220 to 250 are therefore repeated. Otherwise, the practitioner or computer may choose to add an intervention (for example, moving a magnetic tool) which can take place in operation 270. If no intervention is required, or if the intervention is complete, then the study is over. It is understood that image analysis (including reconstruction) may take place between or during multiple steps. Additional steps (not shown) may include deposition of RF energy into the sample.

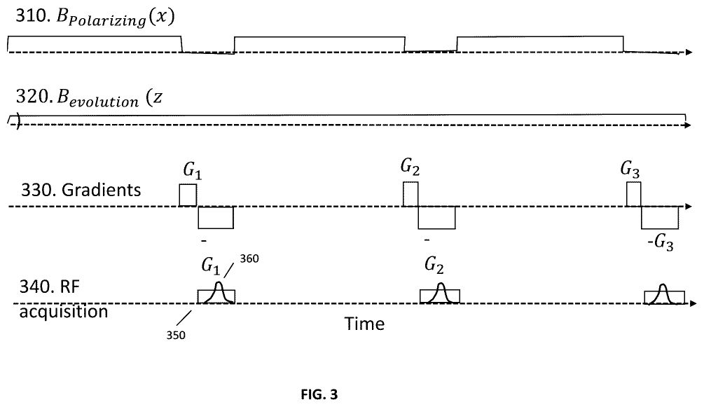

As seen in FIG. 3, a polarizing field 310 may be applied by the polarizing coil (130), for example in the X direction. A smaller evolution field 320 in an orthogonal (BO) direction (i.e., Z-direction as in FIG. 3) may be applied while the polarizing coil is off, or throughout the polarizing field as shown in FIG. 3. Magnetic gradients 330 may be applied when the polarizing field is off. Line 340 shows RF acquisition windows 350 and echoes 360. For purposes of illustration, the magnitude of the polarizing field may be 30 milliTesla, the evolution field magnitude may be 10 milliTesla, the gradient field magnitudes may by 1 milliTesla, and the durations of the polarizing fields and the RF acquisition windows may be 1-10 microseconds.

A novel aspect of the disclosed embodiments is that the short decay times (T1 and T2) may be accommodated by pulsed actuation of the current-carrying coils (130) through (150) in order to generate magnetic field strengths in the 1 to 100 milliTesla range. For example, rapid discharge of a one or more capacitors (containing about 100 microFarads, and charged to 1000 volts from a multi-stage Dickson charge pump or SEPIC-type boost circuit) in source (120) may deliver 100 amperes to a head-sized coil with inductance of 50 microHenries to generate a magnetic field of about 30 milliTeslas for several microseconds. Because of the short duration of the magnetic pulses, and the overall short length of the pulse sequence, cooling of the coils may not be required. As a result of this innovation, the coils of the entire apparatus may be lightweight (e.g., less than several kilograms) and the source 120 may be powered from a small generator or handheld or car battery. In some embodiments, the apparatus may weigh less than 10 kg.

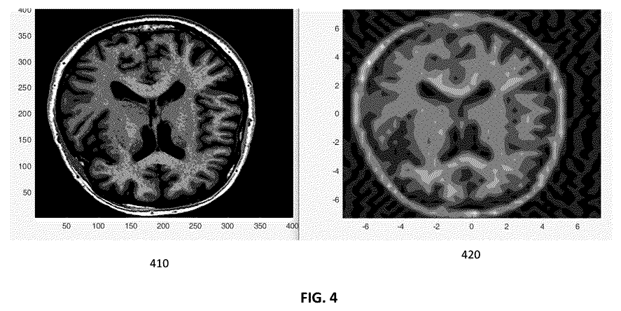

FIG. 4 shows the results of a simulation of EPRI using literature estimates of free electron density and a pulse sequence similar to that of FIG. 3, with a total acquisition time of about less than one second for the entire brain. The presumed distribution of free electrons in a single transverse brain slice is shown 410, and the reconstructed estimate of the free electron density is shown in 420. The images shown in FIG. 4 were obtained using Bloch-equation simulations applied to a model-based reconstruction. The images were obtained with the following assumptions, which represent a non-limiting embodiment of the disclosed embodiments: The echoes are formed by applying a polarizing magnetic field (about 100 milliTesla, lasting for several microseconds, with rising time of few microseconds and decay time of hundreds of nanoseconds) that is orthogonal to the evolution field (10 milliTeslas, corresponding to an electron spin precession frequency of 300 MHZ), followed by a set of opposite gradients (with rising and decay time of 100 nanoseconds), akin to an echo planar imaging (EPI) sequence. An echo gradient sequence is used, rather than the usual method of applying RF pulses to the sample to form and re-form the echoes, to reduce or totally avoid depositing RF energy into the sample. The rapid cessation of the load current in the polarizing coil (assumed to be 20 cm in diameter with 20 turns, for a total inductance of 70 microHenries, with a maximum load voltage of 1000 volts) may be accomplished through a fast switch that applies a high resistance in series with the inductive load. The rise time of the gradient coil magnetic field (with a current of 100 Amps going into a load of about 1 microHenries, yielding about 2 milliTeslas) may be about 100 nanoseconds. One echo occurs every time the polarizing field cycles (about 2 microseconds). The total imaging time for ten slices, each of which contains 1600 pixels, is a few hundred milliseconds.

It is understood that the system may be modular, in which each module may have its own RF and gradient coils, and may also have its own power supplies.

The signal strength per echo will depend on the density of free electrons. According to the publication by Luca Turina, Efthimios M. C. Skoulakis, and Andrew P. Horsfield, PNAS E3524 (2014) entitled: “Electron spin changes during general anesthesia in Drosophila”. If no spin traps are used, the ESR signal corresponds to the electron spin density of 1.5 microMolar, and a free electron concentration of 6 milliMolar can be calculated with the relation of Equation 1:

$${n}_{e} ∼ {k}_{B} T / {μ}_{e} H \qquad ( 1 )$$

The signal can be estimated using the following relations, where Sₑ is the signal from the electrons at an evolution field Bₑ of 10 milliTeslas, Sₚ is the nuclear magnetic resonance signal from protons at Bₚ of 1 Tesla, and Pₑ is the ratio of the polarizing field to the evolution field.

$$\frac{{S}_{e}}{{S}_{p}} ∼ \frac{{μ}_{e}}{{μ}_{p}} {( \frac{{μ}_{e} {B}_{e}}{{μ}_{p} {B}_{p}} )}^{2} \frac{{n}_{e}}{{n}_{p}} {P}_{e} \qquad ( 2 )$$

$$\frac{{S}_{e}}{{S}_{p}} ∼ \frac{{μ}_{e}}{{μ}_{p}} {( \frac{{μ}_{e} {B}_{e}}{{μ}_{p} {B}_{p}} )}^{2} \frac{{n}_{e}}{{n}_{p}} \frac{{V}_{e}}{{V}_{p}} ∼ \frac{1}{2} \qquad ( 3 )$$

While the concentration of free electrons is much smaller than that of protons in water (nₚ˜100 M), the signal electrons produce can be comparable to that of protons. This is due to much higher magnetic moment of electron (due to the higher polarization of electron ensemble, as well as the higher frequency). If spin traps are used, the effective signal will be much higher than from protons.

It is known that many types of insults to the body increase the number of free electrons, so that the invention may be used to monitor insults to the underlying tissues. For example, application of therapeutic radiation can create enough free electrons to destroy tissues. Oxidation-reduction reactions (“redox”) form free electrons, and are involved in many pathological and normal states, as taught for example by: JI Sbodio, SH Snyder, and BD Paul in the article entitled “Redox Mechanisms in Neurodegeneration: From Disease Outcomes to Therapeutic Opportunities”, published in Antioxid Redox Signal. 2019; 30 (11): 1450-1499. doi: 10.1089/ars.2017.7321. Exposure to neurotoxins can also increase free electron density in the brain, as taught by RJ Reiter, LC Manchester, and DX Tan in the article entitled “Neurotoxins: free radical mechanisms and melatonin protection”, published in Curr Neuropharmacol. 2010; 8 (3): 194-210. doi: 10.2174/157015910792246236.

In the heart, free electrons can be generated by ischemia and reperfusion, as taught by JE Baker, CC Felix, GM Olinger, and B Kalyanaraman, in the article entitled “Myocardial ischemia and reperfusion: direct evidence for free radical generation by electron spin resonance spectroscopy”, published in Proc Natl Acad Sci USA. 1988 Apr.;85 (8): 2786-9. doi: 10.1073/pnas.85.8.2786.

Increased free electrons may also be present in normal brain function, as taught by R Franco and MR Vargas in the article entitled “Redox Biology in Neurological Function, Dysfunction, and Aging”, published in Antioxid Redox Signal. 2018; 28 (18): 1583-1586. doi: 10.1089/ars.2018.7509.

Aside from the advantages of low magnetic evolution fields (e.g., 5-10 milliTeslas) related to the pulsed power considerations described above, another motivation for using a relatively low evolution field is that the penetration depth of RF signals emanated from human tissues is dependent on the frequency. At 300 MHZ, the penetration depth is about 10 centimeters. If multiple RF receive coils are used, it should be possible to image the entire human head. MRI systems with superconducting magnets at 5 to 10 Tesla (200-400 MHZ operating frequency) have been able to collect RF signals to image the entire brain, as taught in the publication by P Roschmann (Medical Physics 14, 922 (1987); doi: 10.1118/1.595995), entitled “Radiofrequency penetration and absorption in the human body: Limitations to high-field whole-body nuclear magnetic resonance imaging”.

It is understood that a mounting assembly (for example a helmet) may be required to hold the coils in place during the study and otherwise. It is understood that the tools or particles that may be manipulated during an intervention may be made of magnetizable materials, for example as taught in U.S. Pat. No. 10,290,404B2 patent by Irving Weinberg, entitled “Method and apparatus for non-contact axial particle rotation and decoupled particle propulsion” and related patents, incorporated herein by reference. Another type of intervention would be opening the blood-brain barrier to drugs or genes with pulsed magnetic fields from one or more of the coils of the invention. Such opening has been taught in the publication by S. Jafari et al., “Opening the Blood Brain Barrier with an Electropermanent Magnet System,” published in Pharmaceutics, vol. 14, no. 7, p. 153 Jul. 2022, doi: 10.3390/pharmaceutics14071503. Another type of intervention would be the application of magnetic or electric fields for neuromodulation. Another type of intervention would be the application of magnetic or electric fields for neuroprotection, as taught by Irving Weinberg in the US patent application number U.S. Ser. No. 18/389,437, entitled “Apparatus and method for treating acute central nervous system injuries with prompt application of electromagnetic fields” incorporated by reference.

It is understood that said charging sources may be batteries, adjustable direct current power supplies, alternating power supplies with diodes or other switches (as are sometimes used in voltage multiplier circuits), switching power supplies, or other voltage or current sources. It is understood that said charging sources may be monitored or regulated with Zener diodes, resistive strings, current probes, and/or other sensors to aid in precise operation of the invention.

It is understood that computer 100 may be a controller or control device that may perform the programming of the charging and discharging operations of the disclosed embodiments. It is understood that the electrical pulses applied to the various coils may be more than 1,000 volts, may be more than 1,000 amps, and may be shorter than 10 microseconds in duration. It is understood that some or all of the functions of computer 100 may be incorporated within the power source 120, and that some functions of the computer and power source may be distributed within modules, each module containing some or all coils and amplifiers required to generate magnetic fields and measure RF signals.

It is understood that the invention may be combined with other sensors (for example: optically-pulsed magnetometers, electroencephalographic electrodes, electrocardiography electrodes, or infra-red sensors) to assist in diagnosis.

Those skilled in the art will recognize, upon consideration of the above teachings, that the above exemplary embodiments and the controller may be based upon use of one or more programmed processors programmed with a suitable computer program. However, the disclosed embodiments could be implemented using hardware component equivalents such as special purpose hardware and/or dedicated processors. Similarly, general purpose computers, microprocessor based computers, micro-controllers, optical computers, analog computers, dedicated processors, application specific circuits and/or dedicated hard wired logic may be used to construct alternative equivalent embodiments.

Moreover, it should be understood that control and cooperation of the above-described components may be provided using software instructions that may be stored in a tangible, non-transitory storage device such as a non-transitory computer readable storage device storing instructions which, when executed on one or more programmed processors, carry out he above-described method operations and resulting functionality. In this case, the term “non-transitory” is intended to preclude transmitted signals and propagating waves, but not storage devices that are erasable or dependent upon power sources to retain information.

Those skilled in the art will appreciate, upon consideration of the above teachings, that the program operations and processes and associated data used to implement certain of the embodiments described above can be implemented using disc storage as well as other forms of storage devices including, but not limited to non-transitory storage media (where non-transitory is intended only to preclude propagating signals and not signals which are transitory in that they are erased by removal of power or explicit acts of erasure) such as for example Read Only Memory (ROM) devices, Random Access Memory (RAM) devices, network memory devices, optical storage elements, magnetic storage elements, magneto-optical storage elements, flash memory, core memory and/or other equivalent volatile and non-volatile storage technologies without departing from certain embodiments. Such alternative storage devices should be considered equivalents.

Although the invention has been explained in relation to various embodiments, it is to be understood that many other possible modifications and variations can be made without departing from the spirit and scope of the invention.

1. An apparatus for collecting data about the free electron density of a sample comprising:
at least one array of at least three coils, and
a controller configured to control the at least one array to apply polarizing and gradient magnetic pulses, with pulse durations of less than 10 microseconds per pulse, and an evolution field of less than 15 milliTeslas, are applied to a sample containing free electrons in order to collect information about the distribution and state of the free electrons.

2. The apparatus of claim 1, wherein the at least one array weighs less than 10 kilograms.

3. The apparatus of claim 1, wherein at least one of the pulse durations is less than 2 microseconds per pulse.

4. The apparatus of claim 1, wherein the at least one array is wearable.

5. The apparatus of claim 4, wherein the at least one array is a helmet.

6. The apparatus of claim 1, wherein the controller uses the collected information to form an image.

7. The apparatus of claim 1, wherein the sample is a human brain or a human heart.

8. A method of collecting data about the free electron density of a sample comprising:
providing at least one array of at least three coils, and
applying polarizing and gradient magnetic fields, via the coils, having pulse durations of less than 10 microseconds to the sample in order to collect information about the distribution and state of the free electrons.

9. The method of claim 8, wherein at least one of the pulse durations is less than 2 microseconds per pulse.

10. The method of claim 8, wherein spin traps are introduced into the sample prior to collecting data.

11. The method of claim 8, further comprising forming an image from the collected information.

12. The method of claim 8, further comprising implementing interventions by utilizing coils to collect information about free-electron distribution and state; and
utilizing coils to open a blood-brain barrier, to impel one or more magnetizable tools, or apply neuromodulation or neuroprotection.
